(19) United States
(12) Patent Application Publication
(10) Pub. No.: US 2016/0106641 A1
(43) Pub. Date: Apr. 21, 2016

(54) STABLE COMPOSITIONS FOR WHITENING TEETH AND METHODS OF USING SAME

(71) Applicant: Oraceutical LLC, Lee, MA (US)

(72) Inventor: R. Eric Montgomery, Monterey, MA (US)

(21) Appl. No.: 14/885,350

(22) Filed: Oct. 16, 2015

Related U.S. Application Data
(60) Provisional application No. 62/066,181, filed on Oct. 20, 2014.

(51) Int. Cl. A61K 8/22; A61Q 11/00; A61K 8/34; A61C 17/22; A61K 8/19; A61C 17/20; A61N 5/06; A61K 8/81; A61K 8/55

(52) U.S. Cl. CPC A61K 8/22; A61K 8/8158; A61Q 11/00; A61K 8/345; A61K 8/55; A61K 8/19; A61C 17/20; A61N 5/062; A61C 17/22; A61K 2800/81; A61K 2800/87; A61K 2800/92; A61K 2800/592; A61N 2005/0606

(57) ABSTRACT

A thickened tooth whitening composition in gel form comprises hydrogen peroxide or a hydrogen peroxide precursor, and a thickening polymer or copolymer comprising a pendant sulfonic acid group moiety, preferably hydroxyethyl acrylate/sodium acryloyldimethyltaurate copolymer. Gels in accordance with the invention exhibit an extended shelf life without requiring refrigeration or other special storage conditions. A particularly efficacious manner of method of whitening teeth comprises applying a volume of a gel in accordance with the invention onto the bristles of a toothbrush, applying a volume of a toothpaste onto the toothbrush bristles, and brushing one or more teeth to be whitened with the toothbrush bristles onto which the toothpaste and the thickened tooth whitening composition were applied for a sufficient period to cause the composition and toothpaste to mix together.

### FIELD OF THE INVENTION

The present invention relates to methods and compositions used to whiten teeth, and more particularly to toothbrushing methods utilizing highly stable oxidizing compositions comprising hydrogen peroxide and an oxidation-resistant thickened carrier comprising a 2-acrylamido-2-methylpropane sulfonic acid (AMPS) polymer or copolymer. The inventive compositions thickened with an AMPS polymer or copolymer are found to be unexpectedly stable, particularly with respect to viscosity and hydrogen peroxide concentration over time, such that highly effective tooth whitening gels may be formulated and stored at room temperature for extended periods of time without the need for refrigeration.

### BACKGROUND OF THE INVENTION

As the connections between healthy teeth and gums, and general overall health, have become increasingly evident in the past 100 years, oral care has become an important part of people's daily health maintenance regimens. In the process, a healthy looking smile has become representative of ones level of personal grooming and even social status, with straight, white and well-shaped teeth being promoted in advertising and by cosmetic dentists as an integral part of one's self-image. Over the past 20 years, the availability of tooth whitening products and services has exploded in the marketplace, ranging from low-priced over-the-counter (OTC) self-applied trays, strips, pens, mouthwashes and toothpastes, to expensive professionally applied or monitored products and procedures capable of effectively whitening teeth in as little as 45 minutes. In general, professionally applied products and services administered to a patient in a dental office or other clinical setting are seen to achieve the best teeth whitening results in the shortest amount of time. This is primarily due to the higher concentrations of active ingredients, usually hydrogen peroxide or a hydrogen peroxide precursor, found in professionally applied whitening compositions. These high concentrations, typically above 15% hydrogen peroxide by weight and often as high as 50% hydrogen peroxide by weight, can only be safely administered in a controlled setting where a professionally trained individual can isolate soft tissues from contact with these highly oxidative compositions. Frequent monitoring of a patient's progress over, for instance, a one-hour period is also critical in maintaining a high degree of safety when working with such high hydrogen peroxide concentrations. Optionally, light or heat energy may be applied in conjunction with these strong oxidizing compositions, in order to accelerate the process beyond that which is possible using just the compositions on their own. In general, these professionally-monitored products and services applied in a dental office or clinic will be referred to collectively as in-office or chairside whitening procedures.

In order to safely and effectively apply hydrogen peroxide in an active form to the teeth surfaces during a chairside whitening procedure, a certain degree of flow control must be maintained in order to allow for precise placement of the hydrogen peroxide on the tooth surfaces in need of whitening. In general, hydrogen peroxide is formulated into gel or paste compositions that have sufficient “body” (or in other words, a high enough level of viscosity and/or thixotropy) in order to adhere, cling or otherwise remain in contact with the tooth surfaces without risk of migrating to adjacent soft tissue surfaces such as the crevicular spaces, the lingual and buccal mucosa, and other soft tissue surfaces in the oral cavity. Whitening compositions with sufficient body are also easier to apply using standard clinical applicators such as brushes, syringes, swabs and other similar devices.

While a wide variety of bodying or thickening agents are available, there are very few that are capable of providing increased viscosity in compositions where oxidizing agents are present. This is most often due to instability of the thickening agent in the presence of hydrogen peroxide, and as most thickening agents are polymers or copolymers, they are susceptible to oxidation reactions that can reduce molecular weight and their ability to maintain a consistent viscosity over time. A number of polymers and copolymers have been successfully employed in hydrogen peroxide teeth whitening compositions to provide sufficient viscosity to form thickened gels and pastes. Polymers and copolymers (hereinafter referred to simply as “polymers” or “polymer”) such as carboxypolymethylene (Carbopol®), acrylic acid/C10-C30 alkyl acrylate copolymers (Pemulen®), polyoxyethylene/polyoxypropylene block copolymers (Pluronic®), and hydroxypropylcellulose (Klucel®), amongst others, have been used with varying degrees of success, but generally these polymers exhibit a time- and temperature-dependent decrease in viscosity in the presence of hydrogen peroxide even when composition stabilizers are present.

The relative lack of success in identifying and employing thickening polymers and copolymers is most likely related to a long-standing common wisdom within the dental materials research community that ingredients riot previously employed or approved for oral care or food applications may not be suitable in tooth whitening compositions. However, the polymers and copolymers used in the compositions of the present invention described and claimed herein possess the relatively low order of toxicity required for tooth whitening applications and were not discounted out of hand for use in such compositions in spite of conventional wisdom to the contrary. In fact, the inventive use of such polymers or copolymers, with their exceptional stability profile that is so important to tooth whitening gel performance, will likely reduce the possibility of patient harm (such as tissue damage and ingestion). This should occur at least in part because of the consistency of rheological properties observed over time for these polymers and copolymers and the resulting reduced likelihood of undesirable liquefaction that has been observed in prior art tooth whitening gel compositions. In addition, polymers and copolymers utilized in the present inventive compositions may be employed for their particularly unique properties, such as reduced water solubility for those circumstances in which long term gel contact could lead to “leaching” of a typical water-soluble gel from a delivery device such as a dental tray. Such leaching can lead to composition ingestion and contact with sensitive salt tissues in the oral cavity and digestive tract. Limiting this result of viscosity degradation is a significant and unexpected improvement over the water-soluble prior art compositions.

Depending upon the rate of viscosity decrease observed in stability studies (conducted typically at 25 degrees C. and 60% relative humidity to represent room temperature storage conditions and at 40 degrees C. and 75% relative humidity to represent accelerated or stressed storage conditions), limitations are placed on the expiration dating of a particular composition. The expiration date represents that date beyond which the performance-related properties (such as remaining concentration of hydrogen peroxide and, in particular, viscosity) are observed or predicted to fall below a suitable established range. In order to extend expiration dates, manufacturers often have no choice but to recommend storage of tooth whitening compositions at refrigerated temperatures, which is inconvenient and time-consuming, but allows for the longer product shelf life demanded by dentists and dental distributors because of the difficulty in precisely predicting when a product in inventory may be used or sold.

A number of thickened hydrogen peroxide gels are commercially available for whitening teeth. In order to preserve hydrogen peroxide potency over a reasonable shelf life (e.g., 12 to 18 months), it is necessary to store these known commercial products in a refrigerated state, typically between 2 degrees C. and 8 degrees C. (36 degrees Fahrenheit to 46 degrees Fahrenheit). Opalescence Boost Gel (Ultradent, South Jordan, Utah) is a 45% hydrogen peroxide gel that despite its low pH of approximately 3.0 (which is more favorable for hydrogen peroxide stability than higher pH levels), the product must still be refrigerated to avoid loss of hydrogen peroxide potency and gel viscosity, both of which would lead to poor performance of the product as a tooth whitener. Colgate Visible White Chairside gel (Colgate-Oral Pharmaceuticals, New York, N.Y.), according to information on its MSDS, is a 40-50% hydrogen peroxide gel with a pH between 1.8 and 2.8. and thickened with silicon dioxide. Refrigerated storage is also recommended for this product to avoid premature degradation of gel potency and theological properties. Both of these commercial products require refrigerated storage, which is often inconvenient in the limited space available in a typical dentist's office, and both products require a waiting period for the gels to equilibrate to room temperature before use, which is often not possible or is inconvenient due to tight patient scheduling windows.

For the reasons stated above, there is a need for thickened tooth whitening compositions that have a stable viscosity and stable concentration of active hydrogen peroxide content over an extended period of time when stored at typical room temperatures or non-refrigerated conditions. The present invention addresses that need. What constitutes an “extended period of time” will depend on the manner in which compositions in accordance with the invention are to be used and stored. One convenient way an extended period of time can be defined in the context of particular compositions described herein is a 24 month period during which the viscosity and hydrogen peroxide content decrease less than 20% of their original values when stored at room temperature, as calculated h a regression analysis of the viscosity and hydrogen peroxide compositions aged at 40 degrees C. Alternatively, an extended period of time can also be considered as a 24 week period during which the viscosity and hydrogen peroxide content decrease less than 20% of their original values when stored at 40 degrees C.

### SUMMARY OF THE INVENTION

One aspect of the present invention is directed to methods of utilizing highly storage-stable oxidizing compositions for general bleaching or whitening purposes, but in particular for whitening teeth, comprising (1) hydrogen peroxide or a hydrogen peroxide precursor and (2) a thickening polymer or copolymer comprising a pendant sulfonic acid group contributed by building block monomers such as 2-acrylamido-2-methylpropane sulfonic acid (AMPS). While not being bound to any particular theory, it is speculated that the pendant sulfonic acid moiety on the thickener polymer or copolymer contributes to the stability of the hydrogen peroxide. This is in stark contrast to prior art thickening polymers or copolymers which are not observed to possess such stabilizing abilities and in fact may contribute to more rapid degradation of the hydrogen peroxide in the composition.

In a another aspect, the present invention is directed to a tooth whitening composition comprising (1) hydrogen peroxide at a concentration between about 1 and 15% by weight, (2) a hydrogen peroxide stabilizer, and (3) a thickening polymer or copolymer comprising a pendant sulfonic acid group. A salient property of the composition is its stability for extended periods of time at room temperature.

The present invention is further directed to a method of using these compositions in conjunction with normal toothbrushing.

These and other features, aspects and advantages of the invention will become evident to those skilled in the art from a reading of the present disclosure.

### BRIEF DESCRIPTION OF THE DRAWINGS

The objects of the invention will be better understood from the detailed description of its preferred embodiments which follows below, when taken in conjunction with the accompanying drawings. The following is a brief identification of the drawing figures used in the accompanying detailed description.

FIG. 1 graphically depicts the results of a regression analysis for the change in hydrogen peroxide concentration of a gel formulated in accordance with Working Example No. 1 herein and stored in polypropylene syringes at 40° C. and 75% relative humidity for 24 weeks.

FIG. 2 graphically depicts the results of a regression analysis for the change in pH of the same gel stored under the same conditions.

FIG. 3 graphically depicts the results of a regression analysis for the change in viscosity of the same gel stored under the same conditions.

### DETAILED DESCRIPTION OF THE INVENTION

While this specification concludes with claims particularly pointing out and distinctly claiming certain aspects of the invention, the following description includes details of particular embodiments of the compositions and methods that comprise the claimed subject matter.

All percentages and ratios used hereinafter are by weight of total composition, unless otherwise indicated. All measurements referred to herein are made at 25° C. unless otherwise specified. All percentages, ratios, and levels of ingredients referred to herein are based on the actual amount of the ingredient, and do not include solvents, fillers, or other materials with which the ingredient may be combined as a commercially available product, unless otherwise indicated.

All publications, patent applications, and issued patents mentioned herein are hereby incorporated in their entirety by reference. Citation of any reference is not an admission regarding any determination as to its availability as prior art to the claimed invention.

The highly stable oxidizing compositions of the present invention comprise an oxidizing compound and a thickening polymer containing as pendant sulfonic acid moiety. In addition to these essential components, the inventive compositions may also contain optional components. Both the essential and optional components will he described more fully in the following paragraphs.

Oxidizing Compounds

Oxidizing compounds contemplated to have utility in the formulation of the inventive compositions include hydrogen peroxide, carbamide peroxide, sodium percarbonate, calcium peroxide, PVP-hydrogen peroxide complexes, allyl methacrylate crosspolymer/hydrogen peroxide complexes, and other similar compounds that yield or generate hydrogen peroxide in the presence of water.

Thickening Polymer

Thickening polymers containing a pendant sulfonic acid moiety that are contemplated to have utility in the formulation of the inventive compositions include 2-acrylamido-2-methylpropane sulfonic acid (AMPS) polymers and copolymers. The molecular structure of AMPS is shown below:

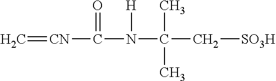

Polymers, copolymers and crosspolymers synthesized from AMPS include hydroxyethyl acrylate/sodium acryloyldimethyltaurate copolymer (Sepinov EMT-10 from Seppic S.A., a division of Air Liquide), ammonium acryloyldimethyltaurate/vinyl pyrrolidone copolymer (Aristoflex AVC, Clariant International LTD), ammonium acryloyldimethyltaurate/beheneth-25 methacrylate crosspolymer (Aristoflex HMB, Clariant International LTD), sodium acrylate/sodium acryloyldimethyltaurate copolymer (a component of Sepigel EG and Simulgel SMS 88, Seppic S.A.), acrylamide/sodium acryloyldimethyltaurate copolymer (a component of Simulgel 600 Simulgel 600 PHA, Seppic S.A.).

Optional Components

Optional components may be added or included in the inventive compositions in order to modify, preserve or enhance the performance or organoleptic characteristics. Optional components include hydrogen peroxide stabilizers, humectants, secondary active ingredients, surface active agents, photoactive compounds, colorants, flavors and sweeteners.

Methods of Use

The inventive compositions can be employed in a number of different methods of whitening teeth, including those intended for application to the teeth of a patient or subject by a dentist or other dental professional, and those intended for application by a patient or subject to his or her own teeth. Tooth whitening methods involving application by a dentist or dental professional are referred to as “chairside” or “in-office” whitening procedures, and typically involve the placement of a tooth whitening gel directly onto the tooth surfaces of a subject while the subject is seated in a dental chair at a dental office, med spa, or other such setting. Tooth whitening methods involving self-application of a tooth whitening composition by a subject are typically referred to as “take-home” or “over-the-counter” whitening procedures. Take-home products may involve the fabrication of a custom-molded tray by a dentist or other dental professional, into which the tooth whitening gel is placed at home by the subject using a syringe, tube or other such container to dispense the gel. Pre-molded or “one-size-fits-all” dental trays, such as those described in U.S. Pat. No. 5,375,654, may be provided “pre-dosed” with a tooth whitening gel for subject or patient convenience. Strips of material can be fashioned from various materials including deformable and non-deformable polymers and waxes, coated with a thin layer of tooth whitening get and applied directly to the tooth surfaces in order to effect whitening. See, for example, U.S. Pat. No. 6,551,579. Dispensing devices with integrated brushes, swabs or dauber applicators, such as those described in U.S. Pat. No. 8;262,390 and Publ. No. US 2014/0011163, to allow for direct application of tooth whitening gels to the teeth surfaces may also be used effectively by a patient or subject. While not intended to impose any limitations as to any particular method of use, it is contemplated that the present inventive compositions, due to their exceptional stability profiles, will be useful in all of the application modes and tooth whitening methods mentioned above, in particular when extended storage periods at room temperature are either desired or required.

Another method of using, the inventive compositions had been discovered to yield unexpected results. By using the inventive compositions during a subject's toothbrushing procedure in combination with toothpaste, it has been found that a significant degree of tooth whitening can be achieved. (The term “toothpaste” as used herein refers to an orally acceptable composition to be applied to the teeth comprising (1) a fluid, semi-fluid or solid carrier, (2) one or more abrasive materials, and (3) optionally one or more therapeutic ingredients such as sodium fluoride, sodium monofluorophosphate, a zinc salt, an antimicrobial compound, and/or other materials capable of effecting a biological change and/or cosmetic improvement to an oral cavity structure.) This inventive method can be practiced by applying a specific amount of one of the inventive compositions onto the bristles of a toothbrush and a separate specific amount of toothpaste onto the bristles of the same toothbrush. The inventive composition may be dispensed onto the toothbrush prior to or after dispensing the toothpaste onto the toothbrush, and the inventive composition may be place side-by-side with the toothpaste on the toothbrush, on top of the toothpaste or underneath the toothpaste. Once the inventive composition and the toothpaste are present on the toothbrush, the subject brushes the teeth normally for a period of time during which the inventive composition and the toothpaste mix together in the oral cavity as a result of the agitation providing by the brushing action. Toothbrushes suitable for use in this method can be manual, electric, sonic, and ultrasonic, or combinations thereof, and can further include light-emitting means such as LEDs or the like to project onto the teeth being brushed actinic light within a predetermined range of wavelengths.

The toothbrushing method above may also be practiced by applying the inventive composition o the tooth surface prior to brushing the teeth with toothpaste. The inventive composition may be applied to the teeth using a brush, swab, conformable dental tray, flexible strip, dispensing pen or other similar applicator (as discussed above) capable of applying the composition to the surfaces of teeth in need of whitening. The compositions may be left in contact with the teeth for a period of time between 10 seconds and 15 minutes to allow the composition oxidizer to penetrate into tooth enamel and dentist. Immediately following the period of time allowed for composition contact with the teeth, the teeth are brushed with toothpaste as they normally would be in a standard home dental hygiene regimen. Using this particular method the inventive composition remaining on the tooth surfaces from the initial application step then mixes with the toothpaste in the subsequent toothbrushing step, providing the tooth whitening benefits described above.

Beyond a twice-daily toothbrushing regimen, a large majority of the population does not have the time or desire to perform any kind of additional oral care procedure to maintain good oral hygiene; therefore the method of tooth whitening using the inventive compositions while brushing the teeth is found to be particularly advantageous.

An unexpected benefit of brushing with toothpaste supplemented by the inventive compositions during brushing is that copious amounts of foam are produced, generally in excess of that provided by using toothpaste alone. The benefits of the additional foam during brushing are improved flavor release and reduced bubble size resulting in finer textured foam. A smaller bubble size is more conducive to penetration of the brushing mixture into smaller spaces on and between teeth, and therefore greater surface area contact for delivery of active ingredients such as the oxidizing agent(s) and, if present, fluoride. While not being bound to any particular theory, it is believed that the finer textured foam and smaller bubbles are a result of the surface activity of the 2-acrylamido-2-methylpropane sulfonic acid (AMPS) polymers and copolymers utilized in the inventive compositions as thickening agents. As will be shown in the following examples, a particularly preferred 2 acrylamido-2 methylpropane sulfonic acid (AMPS) copolymer is the hydroxyethyl acrylate/sodium acryloyldimethyltaurate copolymer (Sepinov EMT-10 from Seppic S.A., a division of Air Liquide), due to its proven low toxicity profile for oral care applications.

The inventive compositions used in this particular method preferably comprise a concentration of one or more oxidizing agents below that which would cause oral soft tissue irritation, generally in the range of from about 1 percent by weight to 30 percent by weight, depending upon the type of oxidizing agents used and the ratio of inventive composition to toothpaste employed in the inventive method. The weight or volume of the inventive composition to toothpaste ratio placed onto the toothbrush is between 1 to 10 to about 10 to 1, preferably between 1 to 2 and 2 to 1 and most preferably 1 to 1. The concentration of oxidizing agent(s) during toothbrushing with the inventive composition/toothpaste mixture is between 0.5 percent and 15 percent, preferably between 1.5 percent and 12 percent and most preferably between 3 percent and 9 percent.

The inventive compositions of this particular method may be packaged in bottles, airless pumps, collapsible squeeze tubes or any other container with a dispensing means suitable for applying the composition onto a toothbrush. The compositions may also be packaged in single dose containers such as packets or syringes. It is also contemplated within the scope of this invention that the composition be packaged in a container wherby a separate transfer applicator, as a swab or spatula, is used to transfer the composition from the container to the toothbrush. Alternatively, the compositions may be contained within a toothbrush comprising a container and a means of transferring the composition from the toothbrush container to the toothbrush brushing head. The compositions may also be packaged in a side-by-side dual chamber container wherby both the oxidizing composition the toothpaste are dispensed simultaneously onto the toothbrush. Such dual chamber containers may also comprise an integrated mixing means, such as a static mixer, to mix the oxidizing agent and toothpaste just prior to being placed onto the toothbrush.

Compositions utilized in this particular method may optionally contain secondary-benefit ingredients such as flavorants, sweeteners, dyes, pigments and secondary active ingredients.

### WORKING EXAMPLES

The following examples further describe and demonstrate embodiments within the scope of the present invention. The examples below are presented solely for the purpose of illustration and are not to be construed as limitations on the invention, as many variations thereof are possible without departing from the scope and spirit of the subject matter claimed herein.

### Example 1

| Ingredient | Wt % (added) | Wt % (net) |
|---|---|---|
| Water (Aqua) | 1.760 | 59.643 |
| Glycerin 99.7% | 5.000 | 5.000 |
| Etidronic acid (60% solution) | 0.500 | 0.300 |
| Potassium Stannate | 0.260 | 0.260 |
| Hydrogen Peroxide (35% solution) | 88.570 | 31.000 |
| Hydroxyethyl Acrylate/Sodium | 3.750 | 3.750 |
| acryloyldimethyltaurate copolymer | | |
| Ammonium Hydroxide | 0.160 | 0.047 |
| (29% solution) (to pH 5.0) | | |
| | 100.000 | 100.000 |

The composition above was manufactured by combining the water, glycerin, hydrogen peroxide, etidronic acid and potassium stannate in a Kynar-coated 2-gallon Ross vacuum agitator mixing vessel and mixing with moderate to high shear agitation until the components were well blended. While continuing to mix, the hydroxyethyl acrylate/sodium acryloyldimethyltaurate copolymer was quickly added and the mixture agitated until the polymer powder was well dispersed and wetted out. The resulting mixture was then transferred to the Ross vacuum agitator, and slow agitation was begun under vacuum to defoam and deaerate the mixture. The agitation was increased and continued under vacuum until the mixture became smooth, relatively clear and free from entrapped bubbles and/or air. The mixture was then adjusted to a pH of about 5.0 with the ammonium hydroxide solution while continuing to mix under a vacuum. The finished mixture, a transparent to translucent gel, was then filled into plastic syringes for further testing.

The finished composition above was tested for viscosity, pH and hydrogen peroxide content as follows:

Viscosity determination—The viscosity of the gel of Example 1, as well as all of the other working examples described herein, was determined by cone-plate viscometry using a Brookfield DVII+ Pro Viscometer (Brookfield Engineering) with cone-plate attachment. Cone-plate spindle 51Z was used and the conditions were 1 rpm spindle speed and temperature was held constant at 25 deg C. using a circulating water bath. The viscosity 24 hours after manufacture was 39,353 centipoise.

pH Determination—The pH of the gel of Example 1, as well as all of the other examples in the present invention, was determined using an Accumet AR15 pH/ISE meter (Fisher Scientific) and a standardized (two-point standardization at pH 4 and pH 7) combination pH probe. All samples were determined in neat form. The pH 24 hours after manufacture was 4.9.

Hydrogen peroxide determination—The hydrogen peroxide content, expressed as weight percent of the total composition weight, was determined using a standard iodometric titration (FMC Product Technical Guide: Hydrogen Peroxide Quantitative Test—Iodometric). The hydrogen peroxide concentration 24 hours after manufacture was 31.7% by weight of the entire composition.

Stability Studies

Several 3cc polypropylene syringes of the gel from Example 1 were placed in a controlled environment storage chamber at 40 degrees C. and 75% relative humidity. The syringes had a barrel with a plunger inserted into one end for applying pressure to the contents of the barrel to dispense them from an opening at the other end. For the first 12 weeks, samples were drawn from the stored syringes and allowed to equilibrate to room temperature overnight. Thereafter, test samples were drawn on a monthly (four week) basis. Viscosity, pH and hydrogen peroxide determination tests were performed as described above with the results reported in Table 1 below.

TABLE 1
Testing results for Example 1 gel stored at 40 degrees C. and
75% relative humidity:

| | | | Hydrogen |
|---|---|---|---|
| Time | Viscosity (cps) | pH | peroxide (wt %) |
| Initial | 39,353 | 4.9 | 31.7 |
| 1 wk | 41,424 | 4.9 | 33.4 |
| 2 wk | 40,181 | 4.9 | 31.6 |
| 3 wk | 40,181 | 5.0 | 30.8 |
| 4 wk | 38,524 | 4.9 | 31.5 |
| 5 wk | 37,696 | 5.0 | 31.5 |
| 6 wk | 38,939 | 4.8 | 31.3 |
| 7 wk | 39,353 | 4.8 | 31.8 |
| 8 wk | 39,767 | 4.9 | 31.2 |
| 9 wk | 36,867 | 4.9 | 31.8 |
| 10 wk | 32,725 | 4.8 | 31.3 |
| 11 wk | 39,353 | 4.9 | 31.3 |
| 12 wk | 37,282 | 4.8 | 31.0 |
| 16 wk | 37,696 | 4.8 | 30.7 |
| 20 wk | 35,625 | 4.5 | 31.8 |
| 24 wk | 29,411 | 4.4 | 30.6 |

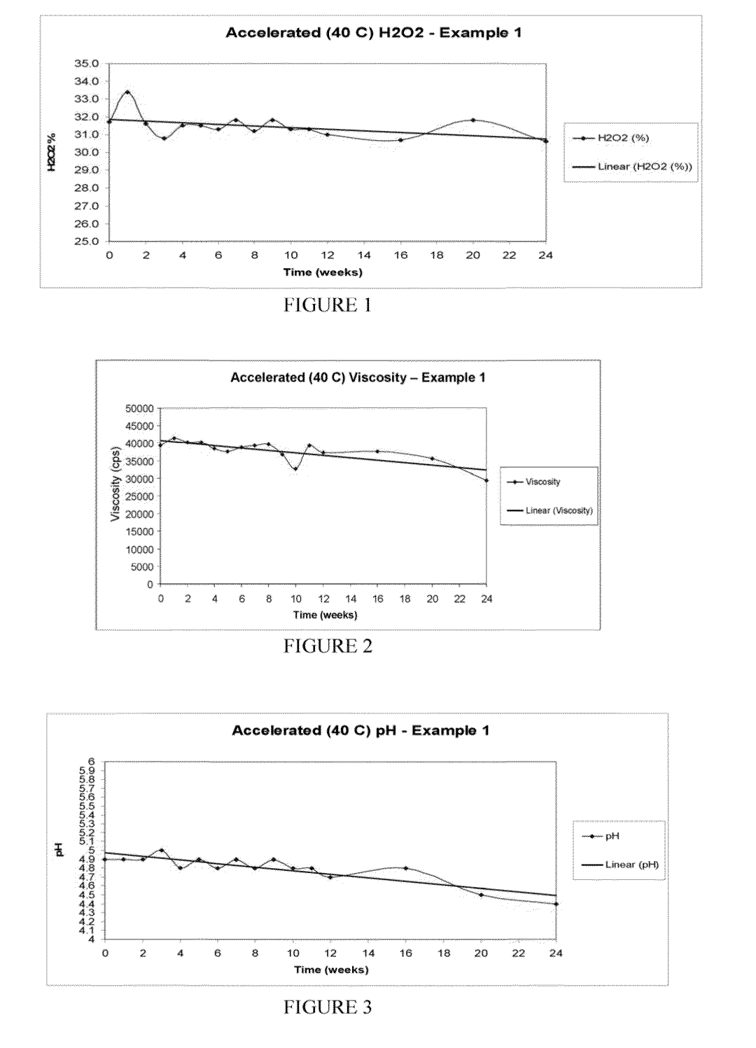

As is clearly evident from the data above, the gel of Example 1 possesses a high degree of stability with respect to viscosity, pH and hydrogen peroxide content. Regression analyses of the above data indicate that all of the physical properties tested will remain within +/−10% range of the initially reported numbers for pH and hydrogen peroxide concentration and within +/−20% of the initially reported numbers for viscosity for at least 24 weeks, which is a high standard of control for performance specifications. Accelerated product aging is typically used to justify expiration dating for new products when room temperature stability testing would delay the introduction of a product to market. Typically, a product stored at 40 degrees C. for 12 weeks can be reliably projected to be stable at room temperature (25 degrees C.) for at least 18 months, and if stored for 24 weeks under the same conditions will be stable at room temperature for at least 24 months. Regression analyses for the changes in hydrogen peroxide concentration, viscosity, and pH in the Example 1 syringe samples stored at 40 degrees C. and 75% relative humidity are shown in FIGS. 1, 2, and 3, respectively.

Clinical Studies

A small pilot clinical study was conducted to determine the tooth whitening efficacy of the gel in Example 1. Five patients were enrolled in the pilot study, all of whom were or good general dental health and had average starting tooth shades between C1 and B3 (arranged according to brightness) on the VITA Classical Shade Guide (Vident Inc., Brea, Calif.). Shades were expressed in terms of numerical values, as follows:

VITA Shade to Numerical Value Conversion Chart (1)

| B1 | A1 | B2 | A2 | D2 | C1 | C2 | D4 |
|---|---|---|---|---|---|---|---|
| 1 | 2 | 3 | 4 | 5 | 6 | 7 | 8 |

VITA Shade to Numerical Value Convasion Chart (2)

| A3 | D3 | B3 | A3.5 | B4 | C3 | A4 | C4 |
|---|---|---|---|---|---|---|---|
| 9 | 10 | 11 | 12 | 13 | 14 | 15 | 16 |

Patients were treated as follows:
1. A cheek retractor was placed in the subject's mouth and soft tissues adjacent to the buccal tooth enamel were isolated using a light-cured resin dam, such as described in U.S. Pat. No. 6,086,370 and No. 6,800,671.
2. The Example 1 gel was dispensed from a syringe directly onto and brushed evenly over the buccal tooth surfaces to be whitened, three of which (teeth #6 to #8) were treated with an alkaline pre-treatment whitening accelerator and three of which (teeth #49 to #11) were not treated with an alkaline pre-treatment accelerator.
3. A contact time of 15 minutes elapsed, at which point the gel was suctioned off the tooth surfaces and gel placement repeated as in (2) above.
4. A second contact time of 15 minutes elapsed, at which point the gel was again suctioned off the tooth surfaces and gel placement repeated as in (2) above.
5. After a final 15 minute gel contact time, the gel was suctioned from the tooth surfaces and all isolation materials were removed.
6. The patient was instructed to rinse with water to remove any residual gel or isolation materials.
7. Final VITA shades were taken and compared to those taken prior to the whitening procedure.

The average VITA shade change for the pre-treatment teeth (#46 to #8) far all of the patients was 5.93, while the average VITA shade change for the non-pre-treatment teeth (#9 to #11) for all of the patients was 5.67. The Example 1 gel was thus observed to whiten teeth effectively with and without an alkaline pre-treatment accelerator.

### Example 2

| Ingredient | Wt % (added) | Wt % (net) |
|---|---|---|
| Water (Aqua) | 5.360 | 45.629 |
| Glycerin 99.7% | 5.000 | 5.000 |
| Etidronic acid (60% solution) | 0.500 | 0.300 |
| Potassium Stannate | 0.260 | 0.260 |
| Hydrogen Peroxide (50% solution) | 90.000 | 45.000 |
| Hydroxyethyl Acrylate/Sodium | 3.750 | 3.750 |
| acryloyldimethyltaurate copolymer | | |
| Ammonium Hydroxide | 0.140 | 0.041 |
| (29% solution) (to pH 5.0) | | |
| | 100.000 | 100.000 |

The composition of Example 2 was manufactured in the same manner as in Example 1, resulting in a translucent gel that was packaged into polypropylene syringes as described above. The Example 2 gel syringes were subjected to a high temperature stress test to determine the gel's ability to maintain functional properties under extremes of temperature. After 24 hours at 85 degrees C. the gel syringe was observed to contain a small number of large bubbles, but viscosity and hydrogen peroxide testing showed little change even after being subjected to these harsh storage conditions.

### Example 3

The comparative stability of the hydrogen peroxide gel of Example 2 was compared to that of a commercially available hydrogen peroxide gel used for tooth whitening in a chairside setting (Opalescence Boost, Ultradent Products, South Jordan, Utah). The hydrogen peroxide concentration of the Boost gel was determined to be 46% using the method previously described, which is similar to the concentration of hydrogen peroxide in the gel of Example 2 (45%). Three samples of both gels (in syringes as discussed above) were placed in a constant temperature oven set to 85 degrees C. for a period of 6 hours, during which the syringes were observed for any movement of the plunger (an indication of poor hydrogen peroxide stability). After 6 hours, all of the plungers of the Boost syringes had been pushed from the barrels by the oxygen gas resulting from the degradation of the hydrogen peroxide into water and oxygen, while the plungers of the syringes with the Example 2 gel were all still intact. Hydrogen peroxide concentrations were not retested for either product, as the loss of the plungers in the Boost samples would have resulted in moisture loss and therefore would have distorted the concentrations. However, viscosity measurements for both the aged accelerated. Boost gel and the Example 2 gel were made using the previously described method. The Boost sample showed a loss of viscosity of 46% (404,000 cps at t=0 and 219,000 at t=6 hours), while the Example 2 gel showed a loss of viscosity of only 15% (42,000 cps at t=0 to 36,000 cps at t=6 hours), demonstrating the superior rheological stability of the inventive composition.

### Example 4

The following composition is designed for use in a patient- or subject-applied tooth whitening method involving a dental tray fabricated by a dentist or dental professional.

| Ingredient | Wt % (added) | Wt % (net) |
|---|---|---|
| Water (Aqua) | 61.400 | 78.562 |
| Glycerin 99.7% | 7.000 | 7.000 |
| Etidronic acid (60% solution) | 0.300 | 0.180 |
| Potassium Stannate | 0.100 | 0.100 |
| Hydrogen Peroxide (35% solution) | 26.000 | 9.100 |
| Hydroxyethyl Acrylate/Sodium | 5.000 | 5.000 |
| acryloyldimethyltaurate copolymer | | |
| Ammonium Hydroxide | 0.200 | 0.058 |
| (29% solution) (to pH 7.0) | | |
| | 100.000 | 100.000 |

### Example 5

The following examples can be used in the toothbrushing methods described above, in conjunction with various packaging modes and/or applicators as described herein.

### Example 5A

| Ingredient | Wt % (added) | Wt % (net) |
|---|---|---|
| Water (Aqua) | 34.890 | 53.340 |
| Glycerin | 30.000 | 30.000 |
| Sodium saccharin | 0.600 | 0.600 |
| Etidronic acid (60% solution) | 0.300 | 0.180 |
| Potassium Stannate | 0.260 | 0.260 |
| Hydrogen Peroxide (35% solution) | 28.200 | 9.870 |
| Hydroxyethyl Acrylate/Sodium | 5.000 | 5.000 |
| acryloyldimethyltaurate copoloymer | | |
| Flavor | 0.750 | 0.750 |
| Potassium hydroxide (45% solution) | to pH 5.5 | to pH 5.5 |
| | 100.000 | 100,000 |

The Example above was packaged in an airless dispensing pump bottle (a 100 ml Mezzo airless pump, supplied by Mega Pumps, Eatontown, N.J. 07724) comprising a dispensing valve that dispensed 0.5 milliliters of the composition each time the actuator valve was depressed. A very controlled dose of the composition can be dispensed utilizing this type of container, thereby controlling within narrow limits the amount of the inventive tooth whitening composition that is used to mix with toothpaste when both the inventive composition and the toothpaste are applied directly onto a toothbrush just prior to brushing.

### Example 5B

| Ingredient | Wt % (added) | Wt % (net) |
|---|---|---|
| Water (Aqua) | 42.233 | 70.210 |
| Glycerin | 10.000 | 10.000 |
| Sodium saccharin | 0.600 | 0.600 |
| Etidronic acid (60% solution) | 0.300 | 0.180 |
| Potassium Stannate | 0.260 | 0.260 |
| Hydrogen Peroxide (35% solution) | 42.857 | 15.000 |
| Hydroxyethyl Acrylate/Sodium | 3.000 | 3.000 |
| acryloyldimethyltaurate copolymer | | |
| Flavor | 0.750 | 0.750 |
| Potassium hydroxide (45% solution) | to pH 5.5 | to pH 5.5 |
| | 100.000 | 100.000 |

This Example 5B was packaged in a dispensing pen comprising a container for holding the composition, a dispensing mechanism comprising an actuator and a movable piston that applies pressure on the composition in the container, a transfer mechanism comprising a transfer means for allowing said composition to flow out of the container in response to pressure applied by said piston, and an applicator means comprising a brush for applying the composition directly to the tooth surface. Other dispensing pen configurations are possible, including those comprising actuators based on a twist or push-button mechanism, or applicator means comprising a foam, fabric, felt, bristle, or other type of material suitable for applying the composition onto a tooth surface. (See Publ. No. US 2014/0011163.) Once applied to the tooth surface, the composition is then allowed to remain in contact with the tooth surface for a period of time and thereafter the residual composition on the tooth surface is mixed with toothpaste during a subsequent normal toothbrushing procedure utilizing toothpaste and a toothbrush. In order to benefit from the combined benefits of the inventive composition and the toothpaste used during the toothbrushing process, it is preferred that the toothbrushing regimen be started while at least traces of the inventive composition are still present on the tooth surface. In general, toothbrushing should begin at a time less than 15 minutes after applying the inventive composition to the tooth surfaces, preferably less than 5 minutes after applying the inventive composition to the tooth surfaces and most preferably less than 60 seconds after applying the inventive composition to the tooth surfaces.

### Example 5C

| Ingredient | Wt % (added) | Wt % (net) |
|---|---|---|
| Water (Aqua) | 32.793 | 60.650 |
| Glycerin | 10.000 | 10.000 |
| Sodium saccharin | 0.600 | 0.600 |
| Hydrogen Peroxide (35% solution) | 42.857 | 15.000 |
| Hydroxyethyl Acrylate/Sodium | 3.000 | 3.000 |
| acryloyldimethyltaurate copolymer | | |
| Flavor | 0.750 | 0.750 |
| Potassium hydroxide (45% solution) | to pH 5.5 | to pH 5.5 |
| | 100.000 | 100.000 |

The composition in this Example 5C was manufactured by combining the water. glycerin, hydrogen peroxide, and sodium saccharin in a Kynar-coated 2-gallon Ross mixing vessel as discussed above and mixing with moderate to high shear agitation until the components were well blended. While continuing to mix, the hydroxyethyl acrylate/sodium acryloyldimethyltaurate copolymer was quickly added and the mixture agitated until the polymer powder was well dispersed and wetted out. The resulting mixture was then transferred to the Ross vacuum agitator, and slow agitation was begun under vacuum to deaerate the mixture. The agitation was increased and continued under vacuum until the mixture became smooth, relatively clear and free from entrapped bubbles and/or air. The mixture was then adjusted to a pH of about 5.5 with a small amount of potassium hydroxide solution while continuing to mix under a vacuum. The flavor was then added and mixed under a mild vacuum (approximately 20 inches Hg) to avoid entrapping air without evaporating the flavor. The finished mixture, a translucent gel, was then filled into either collapsible squeeze tubes or airless pump dispensers for clinical and consumer use testing.

### Example 6

A clinical study utilizing the composition of Example 5A was conducted to determine the additive tooth whitening effect of combining the inventive compositions with toothpaste during 2-minute toothbrushing sessions of 17 subjects over 30 days. Subjects were instructed to dispense equal amounts of the inventive composition and the toothpaste they normally use onto their regular toothbrush, and to brush as they normally would for 2 minutes. Subjects were specifically instructed to brush the upper buccal tooth surfaces for the first 30 seconds of the brushing routine, followed by brushing the lower buccal tooth surfaces for the second 30 seconds of the brushing routine. The lingual tooth surfaces were then brushed for the remaining 60 seconds of the prescribed toothbrushing session. For each subject, a baseline tooth shade color was taken at the initial consultation by a professional evaluator using a VITA Bleaching Shade Guide Master (VITA Zahnfabrik), as well as a final tooth shade color following the end of the 30 day trial.

The following results were observed and recorded:
1. None of the subjects experienced any tooth or gum sensitivity at any time during the 30 day trial.
2. Four subjects reported noticeably whiter teeth after the first day.
3. Six subjects reported noticeably whiter teeth after the second day.
4. All subjects reported noticeably whiter teeth following the 30 day trial period.
5. The professionally-measured average tooth shade color change for the 17 subjects following the 30 day trial was 5.5 shades, with a minimum shade change of 2.0 shades and a maximum shade change of 10.0 shades.

The following Table 2 contains a summary of the approximate ranges of concentrations (on a weight percent basis) for various components that may be present in the inventive compositions useful in the practice of the brushing application methods described above.

TABLE 2

| Component | Percent (w/w) |
|---|---|
| Water (or other carrier) | 1-98 |
| Hydrogen peroxide (or precursor) | 1-30 |
| Sulfonic acid polymer/copolymer | 0.1-10 |
| Stabilizers | 0.01-50 |
| Secondary active ingredients | 0-20 |
| Humectants | 0-95 |
| Colorants | 0-5 |
| Flavorants | 0-3 |
| Sweeteners | 0-70 |

### SUMMARY

It will be understood that the embodiments of the invention described above can be modified in myriad ways other than those specifically discussed without departing from the scope of the invention. Those skilled in the art will readily recognize that only selected preferred embodiments of the invention have been described, and it will be understood that various changes and modifications can be made other than those specifically mentioned above without departing from the spirit and scope of the invention, which is defined solely by the claims that follow.

What is claimed is:

1. A method of whitening teeth comprising:
applying a volume of a thickened tooth whitening composition comprising an oxidizing agent and a 2-acrylamido-2-methylpropane sulfonic acid polymer or copolymer onto bristles of a toothbrush;
applying a volume of a toothpaste onto the toothbrush bristles; and
brushing one or more teeth to be whitened with the toothbrush bristles onto which the toothpaste and the thickened tooth whitening composition were applied for a sufficient period to cause the composition and toothpaste to mix together.

2. A method of whitening teeth as in claim 1 wherein the toothbrush is manual, electric, sonic, or ultrasonic, or combinations thereof.

3. A method of whitening teeth as in claim 3, wherein the toothbrush includes light-emitting means for projecting actinic light within a predetermined range of wavelengths onto the teeth being brushed.

4. A method of whitening teeth as in claim 1 wherein the volume of the toothpaste is between 0.1 milliliters and 3.0 milliliters.

5. A method of whitening teeth as in claim 1 wherein the volume of the thickened tooth whitening composition is between 0.1 milliliters and 3.0 milliliters.

6. A method of whitening teeth as in claim 1 wherein the ratio of the volume of the toothpaste to the thickened tooth whitening composition is between 1:10 and 10:1.

7. A method of whitening teeth as in claim 1 wherein the oxidizing agent is selected from the group comprising hydrogen peroxide, hydrogen peroxide precursors, carbamide peroxide, sodium percarbonate, sodium perborate, sodium chlorite, and peroxyacids.

8. A method of whitening teeth as in claim 1 wherein the oxidizing agent in the thickened tooth whitening composition is present in a concentration between 1 percent and 15 percent b weight of the overall composition.

9. A method of whitening teeth comprising:
applying to one or more teeth to be whitened a volume of a thickened tooth whitening composition consisting essentially of an oxidizing agent and a 2-acrylamido-2-methylpropane sulfonic acid polymer or copolymer;
applying a volume of a toothpaste onto bristles of a toothbrush; and
brushing one or more of the teeth having, the tooth whitening composition thereon with the toothbrush bristles onto which the toothpaste was applied for a sufficient period to cause the composition and the toothpaste to mix together.

10. A method of whitening teeth as in claim 9 wherein the brushing step is performed between 10 seconds and 15 minutes after the applying step.

11. A thickened tooth whitening composition comprising (1) hydrogen peroxide concentration between about 1 and 15% by weight, (2) a hydrogen peroxide stabilizer, and (3) a thickening polymer or copolymer comprising a pendant sulfonic acid group, wherein said composition maintains a stable hydrogen peroxide concentration and viscosity for an extended period of time without refrigeration.

12. A thickened tooth whitening composition as in claim 11, wherein the extended period of time is a 24 month period during which the viscosity and hydrogen peroxide content decrease less than 20% of their original values when stored at room temperature.

13. A thickened tooth whitening composition comprising (1) hydrogen peroxide or a hydrogen peroxide precursor and (2) a thickening polymer or copolymer comprising a pendant sulfonic acid group moiety, wherein said composition maintains a stable hydrogen peroxide concentration and viscosity for an extended period of time without refrigeration.

14. A thickened tooth whitening composition as in claim 13, wherein the extended period of time is a 24 month period during which the viscosity and hydrogen peroxide content decrease less than 20% of their original values when stored at room temperature.

15. A thickened tooth whitening composition as in claim 13 wherein the pendant sulfonic acid moiety includes 2-acrylamido-2-methylpropane sulfonic acid (AMPS) polymers and copolymers having the following molecular structure:

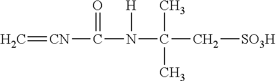

16. A thickened tooth whitening composition as in claim 15 wherein the pendant sulfonic acid moiety is selected from the group of polymers, copolymers and crosspolymers synthesized from AMPS, including (1) hydroxyethyl acrylate/sodium acryloyldimethyltaurate copolymer, (2) ammonium acryloyldimethyltaurate/vinyl pyrrolidone copolymer, (3) ammonium acryloyldimethyltaurate/beheneth-25 methacrylate crosspolymer, (4) sodium acrylate/sodium acryloyldimethyltaurate copolymer, (5) acrylamide/sodium acryloyldimethyltaurate copolymer.

17. A thickened tooth whitening composition as in claim 16 in the form of a gel with a pH of at least about 5, the composition comprising (1) water, (2) glycerin, (3) etidronic acid, (4) potassium stannate, (5) hydrogen peroxide, and (6) hydroxyethyl acrylate/sodium acryloyldimethyltaurate copolymer.

18. A thickened tooth whitening composition as in claim 17, wherein the hydroxyethyl acrylate/sodium acryloyldimethyltaurate copolymer comprises between about 3.0 weight % to about 7.0 weight % of the composition.
